(19) United States
(12) Patent Application Publication
(10) Pub. No.: US 2011/0081726 A1
(43) Pub. Date: Apr. 7, 2011

(54) Signal Dropout Detection and/or Processing in Analyte Monitoring Device and Methods

(75) Inventor: Glenn Howard Berman, Alameda, CA (US)

(73) Assignee: Abbott Diabetes Care Inc., Alameda, CA (US)

(21) Appl. No.: 12/895,790

(22) Filed: Sep. 30, 2010

Related U.S. Application Data
(60) Provisional application No. 61/247,522, filed on Sep. 30, 2009.

(51) Int. Cl. G01N 33/66; G06F 19/00

(52) U.S. Cl. 436/95; 702/104

(57) ABSTRACT

Methods and devices for receiving a plurality of signals from a transcutaneously positioned analyte sensor, receiving a reference data, calibrating the analyte sensor based on the received reference data to generate calibrated sensor data, detecting a change in the level of the received plurality of signals from the analyte sensor exceeding a predetermined threshold level within a preset time period after calibrating the analyte sensor, and generating an output signal based on the detected change are provided. Systems and kits for performing the same are also provided.

### RELATED APPLICATION

The present application claims the benefit of U.S. Provisional Application No. 61/247,522 filed Sep. 30, 2009, entitled “Signal Dropout Detection and/or Processing in Analyte Monitoring Device and Methods”, the disclosure of which is incorporated herein by reference for all purposes.

### BACKGROUND

There are a number of instances when it is desirable or necessary to monitor the concentration of an analyte, such as glucose, lactate, or oxygen, for example, in bodily fluid of a body. For example, it may be desirable to monitor high or low levels of glucose in blood or other bodily fluid that may be detrimental to a human. In a healthy human, the concentration of glucose in the blood is maintained between about 0.8 and about 1.2 mg/mL by a variety of hormones, such as insulin and glucagons, for example. If the blood glucose level is raised above its normal level, hyperglycemia develops and attendant symptoms may result. If the blood glucose concentration falls below its normal level, hypoglycemia develops and attendant symptoms, such as neurological and other symptoms, may result. Both hyperglycemia and hypoglycemia may result in death if untreated. Maintaining blood glucose at an appropriate concentration is thus a desirable or necessary part of treating a person who is physiologically unable to do so unaided, such as a person who is afflicted with diabetes mellitus.

Devices have been developed for continuous or automatic monitoring of analytes, such as glucose, in bodily fluid such as in the blood stream or in interstitial fluid. Some of these analyte measuring devices are configured so that at least a portion of the devices are positioned below a skin surface of a user, e.g., in a blood vessel or in the subcutaneous tissue of a user.

Following the sensor insertion, the resulting potential trauma to the skin and/or underlying tissue, for example, by the sensor introducer and/or the sensor itself, may, at times, result in instability of signals detected or monitored by the sensor. This may occur in a number of analyte sensors, but not in all cases. This instability is characterized by a decrease in the sensor signal, and when this occurs, generally, the corresponding analyte levels monitored may exhibit inaccuracies such as false readings associated with such signal dropout occurrences. Certain instability of the sensor signals may be attributed to signal dropout occurrences that may be a result of a physiological response to the introduction of the analyte sensor to the subcutaneous tissue, incorrect positioning or dislodgement of the analyte sensor or other error conditions which do not accurately reflect the monitored analyte level. Additionally, during the time period of sensor use, signal dropout occurrences may additionally be detected.

### INCORPORATION BY REFERENCE

Patents, applications and/or publications described herein, including the following patents, applications and/or publications are incorporated herein by reference for all purposes: U.S. Pat. Nos. 4,545,382, 4,711,245, 5,262,035, 5,262,305, 5,264,104, 5,320,715, 5,356,786, 5,509,410, 5,543,326, 5,593,852, 5,601,435, 5,628,890, 5,820,551, 5,822,715, 5,899,855, 5,918,603, 6,071,391, 6,103,033, 6,120,676, 6,121,009, 6,134,461, 6,143,164, 6,144,837, 6,161,095, 6,175,752, 6,270,455, 6,284,478, 6,299,757, 6,338,790, 6,377,894, 6,461,496, 6,503,381, 6,514,460, 6,514,718, 6,540,891, 6,560,471, 6,579,690, 6,591,125, 6,592,745, 6,600,997, 6,605,200, 6,605,201, 6,616,819, 6,618,934, 6,650,471, 6,654,625, 6,676,816, 6,730,200, 6,736,957, 6,746,582, 6,749,740, 6,764,581, 6,773,671, 6,881,551, 6,893,545, 6,932,892, 6,932,894, 6,942,518, 7,041,468, 7,167,818, and 7,299,082, U.S. Published Application Nos. 2004/0186365, 2005/0182306, 2006/0025662, 2006/0091006, 2007/0056858, 2007/0068807, 2007/0095661, 2007/0108048, 2007/0199818, 2007/0227911, 2007/0233013, 2008/0066305, 2008/0081977, 2008/0102441, 2008/0148873, 2008/0161666, 2008/0267823, and 2009/0054748, U.S. patent application Ser. Nos. 11/461,725, 12/131,012, 12/393,921, 12/242,823, 12/363,712, 12/495,709, 12/698,124, 12/698,129, 12/714,439, 12/794,721, 12/807,278, 12/842,013, and 12/871,901, and U.S. Provisional Application Nos. 61/238,646, 61/246,825, 61/247,516, 61/249,535, 61/317,243, 61/345,562, and 61/361,374.

### SUMMARY

In view of the foregoing, in accordance with embodiments of the present disclosure there are provided methods and devices for receiving a plurality of signals from a transcutaneously positioned analyte sensor, receiving a reference data, calibrating the analyte sensor based on the received reference data to generate calibrated sensor data, detecting a change in the level of the received plurality of signals from the analyte sensor exceeding a predetermined threshold level within a preset time period after calibrating the analyte sensor, and generating an output signal based on the detected change.

In certain embodiments, there are provided methods and devices for receiving a plurality of signals from a transcutaneously positioned analyte sensor, outputting an indicator associated with the received plurality of signals from the sensor, detecting a change in the level of the received plurality of signals from the analyte sensor exceeding a predetermined threshold level within a preset time period, confirming an adverse condition based on the detected change in the level of the received plurality of signals from the sensor, and modifying a portion of the outputted indicator based on the confirmed adverse signal condition.

Systems and kits for implementing the methods described above are also contemplated.

These and other features, objects and advantages of the present disclosure will become apparent to those persons skilled in the art upon reading the details of the present disclosure as more fully described below.

### BRIEF DESCRIPTION OF THE DRAWINGS

FIG. 1 shows a block diagram a data monitoring and management system usable with a continuous analyte monitoring system in certain embodiments;

FIG. 2 shows a block diagram of a transmitter unit of the data monitoring and management system of FIG. 1 in certain embodiments;

FIG. 3 shows a block diagram of the receiver/monitor unit of the data monitoring and management system of FIG. 1 in certain embodiments;

FIG. 4 shows a schematic diagram of an analyte sensor usable with a continuous analyte monitoring system in certain embodiments;

FIGS. 5A and 5B show perspective and cross sectional views, respectively, of an analyte sensor usable with a continuous monitoring system in certain embodiments;

FIG. 6 is a flowchart illustrating a signal dropout detection and processing routine in certain embodiments;

FIG. 7 is a flowchart illustrating a signal dropout detection and processing routine in certain embodiments; and

FIG. 8 is a flowchart illustrating a signal dropout detection and processing routine in certain embodiments.

### DETAILED DESCRIPTION

Before the present disclosure is described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the present disclosure. The upper and lower limits of these smaller ranges may independently be included in the smaller ranges is also encompassed within the present disclosure, subject to any specifically excluded limit in the stated range. Where the stated range includes one or both of the limits, ranges excluding either or both of those included limits are also included in the present disclosure.

Generally, embodiments of the present disclosure relate to methods and devices for detecting at least one analyte, such as glucose, in body fluid. Embodiments relate to the continuous and/or automatic in vivo monitoring of the level of one or more analytes using a continuous analyte monitoring system that includes an analyte sensor for the in vivo detection of an analyte, such as glucose, lactate, and the like, in a body fluid. Embodiments include wholly implantable analyte sensors and analyte sensors in which only a portion of the sensor is positioned under the skin and a portion of the sensor resides above the skin, e.g., for contact to a control unit, transmitter, receiver, transceiver, processor, etc. At least a portion of a sensor may be, for example, subcutaneously positionable in a patient for the continuous or semi-continuous monitoring of a level of an analyte in a patient's interstitial fluid. For the purposes of this description, semi-continuous monitoring and continuous monitoring will be used interchangeably, unless noted otherwise.

The sensor response may be correlated and/or converted to analyte levels in blood or other fluids. In certain embodiments, an analyte sensor may be positioned in contact with interstitial fluid to detect the level of glucose, which detected glucose may be used to infer the glucose level in the patient's bloodstream. Analyte sensors may be insertable into a vein, artery, or other portion of the body containing fluid. Embodiments of the analyte sensors of the subject disclosure may be configured for monitoring the level of the analyte over a time period which may range from minutes, hours, days, weeks, or longer.

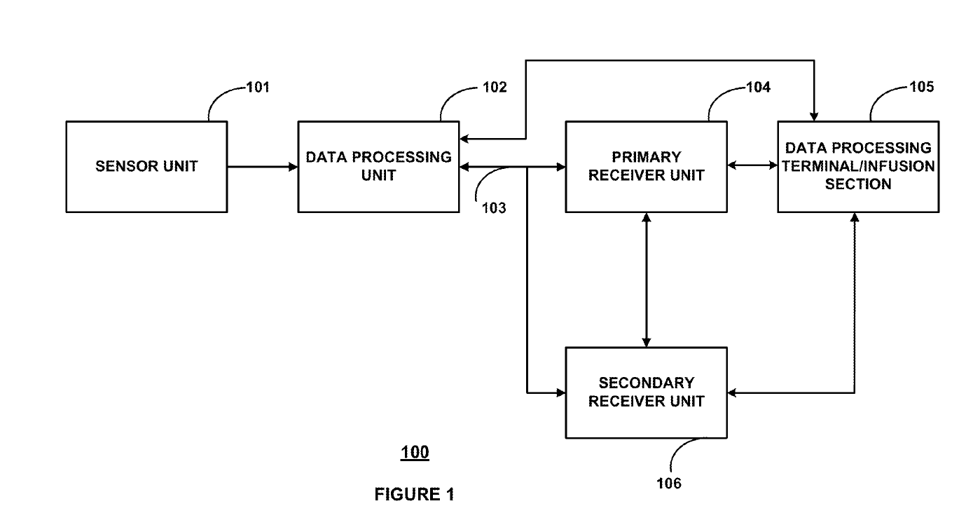

FIG. 1 shows a data monitoring and management system such as, for example, an analyte (e.g., glucose) monitoring system 100 in accordance with certain embodiments. Embodiments of the subject disclosure are further described primarily with respect to glucose monitoring devices and systems, and methods of glucose detection, for convenience only and such description is in no way intended to limit the scope of the invention. It is to be understood that the analyte monitoring system may be configured to monitor a variety of analytes instead of or in addition to glucose, e.g., at the same time or at different times.

Analytes that may be monitored include, but are not limited to, acetyl choline, amylase, bilirubin, cholesterol, chorionic gonadotropin, creatine kinase (e.g., CK-MB), creatine, creatinine, DNA, fructosamine, glucose, glutamine, growth hormones, hormones, ketone bodies, lactate, oxygen, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In those embodiments that monitor more than one analyte, the analytes may be monitored at the same or different times.

In certain embodiments, the analyte monitoring system 100 includes a sensor 101, a data processing unit 102 connectable to the sensor 101, and a primary receiver unit 104 which is configured to communicate with the data processing unit 102 via a communication link 103. In certain embodiments, the primary receiver unit 104 may be further configured to transmit data to a data processing terminal 105 to evaluate or otherwise process or format data received by the primary receiver unit 104. The data processing terminal 105 may be configured to receive data directly from the data processing unit 102 via a communication link which may optionally be configured for bi-directional communication. Further, the data processing unit 102 may include a transmitter or a transceiver to transmit and/or receive data to and/or from the primary receiver unit 104 and/or the data processing terminal 105 and/or optionally the secondary receiver unit 106.

Also shown in FIG. 1 is an optional secondary receiver unit 106 which is operatively coupled to the communication link and configured to receive data transmitted from the data processing unit 102. The secondary receiver unit 106 may be configured to communicate with the primary receiver unit 104, as well as the data processing terminal 105. The secondary receiver unit 106 may be configured for bi-directional wireless communication with each of the primary receiver unit 104 and the data processing terminal 105.

As discussed in further detail below, in certain embodiments the secondary receiver unit 106 may be a de-featured receiver as compared to the primary receiver, i.e., the secondary receiver may include a limited or minimal number of functions and features as compared with the primary receiver unit 104. As such, the secondary receiver unit 106 may include a smaller (in one or more, including all, dimensions), compact housing or be embodied in a device such as a wrist watch, arm band, etc., for example. Alternatively, the secondary receiver unit 106 may be configured with the same or substantially similar functions and features as the primary receiver unit 104. The secondary receiver unit 106 may include a docking portion to be mated with a docking cradle unit for placement by, e.g., the bedside for nighttime monitoring, and/or a bi-directional communication device. A docking cradle may recharge a power supply.

Only one sensor 101, data processing unit 102 and data processing terminal 105 are shown in the embodiment of the analyte monitoring system 100 illustrated in FIG. 1. However, it will be appreciated by one of ordinary skill in the art that the analyte monitoring system 100 may include more than one sensor 101 and/or more than one data processing unit 102, and/or more than one data processing terminal 105. Multiple sensors may be positioned in a patient for analyte monitoring at the same or different times. In certain embodiments, analyte information obtained by a first positioned sensor may be employed as a comparison to analyte information obtained by a second sensor. This may be useful to confirm or validate analyte information obtained from one or both of the sensors. Such redundancy may be useful if analyte information is contemplated in critical therapy-related decisions.

The analyte monitoring system 100 may be a continuous monitoring system or semi-continuous. In a multi-component environment, each component may be configured to be uniquely identified by one or more of the other components in the system so that communication conflict may be readily resolved between the various components within the analyte monitoring system 100. For example, unique identification codes (IDs), communication channels, and the like, may be used.

In certain embodiments, the sensor 101 is physically positioned in and/or on the body of a user whose analyte level is being monitored. The sensor 101 may be configured to continuously or semi-continuously sample the analyte level of the user automatically (without the user initiating the sampling), based on a programmed interval such as, for example, but not limited to, once every minute, once every 5 minutes and so on, and convert the sampled analyte level into a corresponding signal for transmission by the data processing unit 102. The data processing unit 102 is coupleable to the sensor 101 so that both devices are positioned in or on the user's body, with at least a portion of the analyte sensor 101 positioned transcutaneously. The data processing unit may include a fixation element such as adhesive or the like to secure it to the user's body.

A mount or mounting unit (not shown) attachable to the user and mateable with the unit 102 may be used. For example, a mount may include an adhesive surface. The data processing unit 102 performs data processing functions, where such functions may include but are not limited to, filtering and encoding of data signals, each of which corresponds to a sampled analyte level of the user, for transmission to the primary receiver unit 104 via the communication link 103. In one embodiment, the sensor 101 or the data processing unit 102 or a combined sensor/data processing unit may be wholly implantable under the skin layer of the user.

In certain embodiments, the primary receiver unit 104 may include a signal interface section including a radio frequency (RF) receiver and an antenna that is configured to communicate with the data processing unit 102 via the communication link 103, and a data processing section for processing the received data from the data processing unit 102 such as data decoding, error detection and correction, data clock generation, data bit recovery, etc., or any combination thereof.

In operation, the primary receiver unit 104 in certain embodiments is configured to synchronize with the data processing unit 102 to uniquely identify the data processing unit 102, based on, for example, an identification information of the data processing unit 102, and thereafter, to continuously or semi-continuously receive signals transmitted from the data processing unit 102 associated with the monitored analyte levels detected by the sensor 101. Referring again to FIG. 1, the data processing terminal 105 may include a personal computer, a portable computer such as a laptop or a handheld device (e.g., personal digital assistants (PDAs), telephone such as a cellular phone (e.g., a multimedia and Internet-enabled mobile phone such as an iPhone®, a Blackberry®, a Palm® based device or similar phone), mp3 or other media player, pager, and the like), or a drug delivery device, each of which may be configured for data communication with the receiver via a wired or a wireless connection. Additionally, the data processing terminal 105 may further be connected to a data network (not shown) for storing, retrieving, updating, and/or analyzing data corresponding to the detected analyte level of the user.

The data processing terminal 105 may include an infusion device such as an insulin infusion pump or the like, which may be configured to administer insulin to patients, and which may be configured to communicate with the primary receiver unit 104 for receiving, among others, the measured analyte level. Alternatively, the primary receiver unit 104 may be configured to have an infusion device integrated therein so that the primary receiver unit 104 is configured to administer insulin (or other appropriate drug) therapy to patients, for example, by administering and modifying basal profiles, as well as by determining appropriate boluses for administration based on, among others, the detected analyte levels received from the data processing unit 102. An infusion device may be an external device or an internal device (wholly implantable in a user).

In certain embodiments, the data processing terminal 105, which may include an insulin pump, may be configured to receive the analyte signals from the data processing unit 102, and thus, incorporate the functions of the primary receiver unit 104 including data processing for managing the patient's insulin therapy and analyte monitoring. In certain embodiments, the communication link 103 as well as one or more of the other communication interfaces shown in FIG. 1, may use one or more of: an RF communication protocol, an infrared communication protocol, a Bluetooth® enabled communication protocol, an 802.11x wireless communication protocol, or an equivalent wireless communication protocol which would allow secure, wireless communication of several units (for example, per HIPPA requirements), while avoiding potential data collision and interference.

In aspects of the present disclosure, one or more of the data processing unit 102, receiver unit 104/106, or the data processing terminal 105 may include a wired or wireless communication interface, port or connector to communicate or transfer data or information. For example, each of these components of the analyte monitoring system 100 may include a USB connector or a corresponding USB port provided, for example, on the respective housing to wired connection. Alternatively, an RF transceiver chip or infrared communication port may be provided in one or more of the data processing unit 102, receiver unit 104/106, or the data processing terminal 105 to allow for wireless data transfer therebetween.

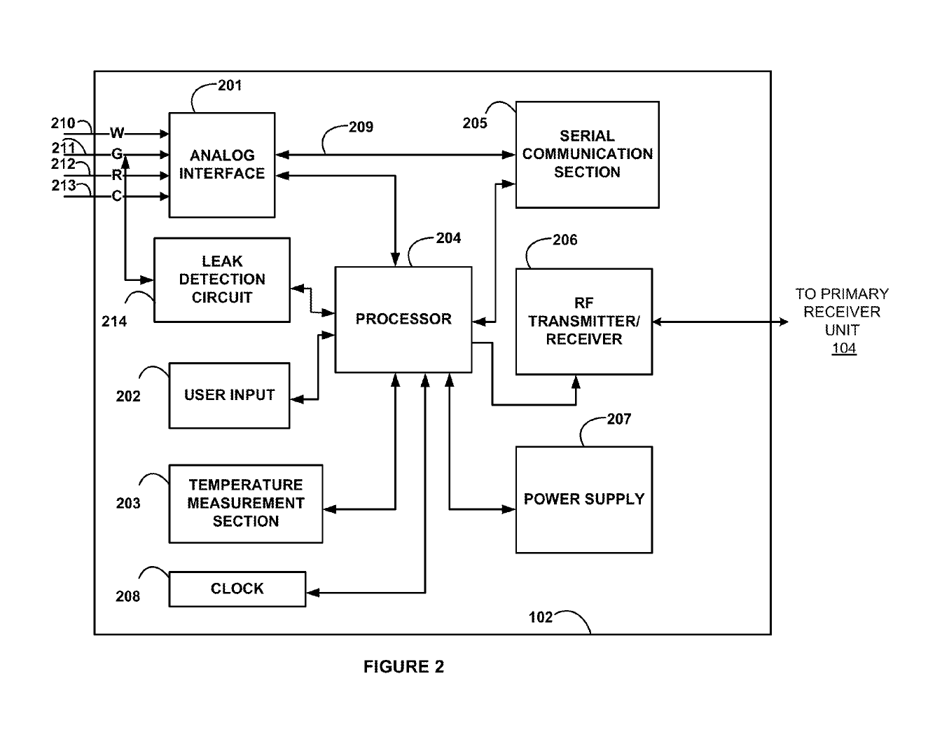

FIG. 2 shows a block diagram of an embodiment of a data processing unit of the data monitoring and detection system shown in FIG. 1. User input 202 and/or interface components may be included or a data processing unit may be free of user input 202 and/or interface components. In certain embodiments, a processor 204, which may include one or more application-specific integrated circuits (ASIC) may be used to implement one or more functions or routines associated with the operations of the data processing unit 102 (and/or receiver unit) using for example one or more state machines and buffers.

As can be seen in the embodiment of FIG. 2, the sensor unit 101 (FIG. 1) includes four contacts, three of which are electrodes—working electrode (W) 210, reference electrode (R) 212, and counter electrode (C) 213, each operatively coupled to the analog interface 201 of the data processing unit 102. This embodiment also shows optional guard contact (G) 211. Fewer or greater electrodes may be employed. For example, the counter and reference electrode functions may be served by a single counter/reference electrode, there may be more than one working electrode and/or reference electrode and/or counter electrode, etc.

Referring still to FIG. 2, processor 204 of data processing unit 102, in certain embodiments, may be coupled to various components of data processing unit 102. Such components include a leak detection circuit 214, temperature measurement section 203, clock 208, serial communication section 205 and power supply 207. Further, the data processing unit 102 may include an RF transmitter/receiver 206, such as an RF transceiver, which may be configured for bi-direction communication with a receiver unit, such as primary receiver unit 104.

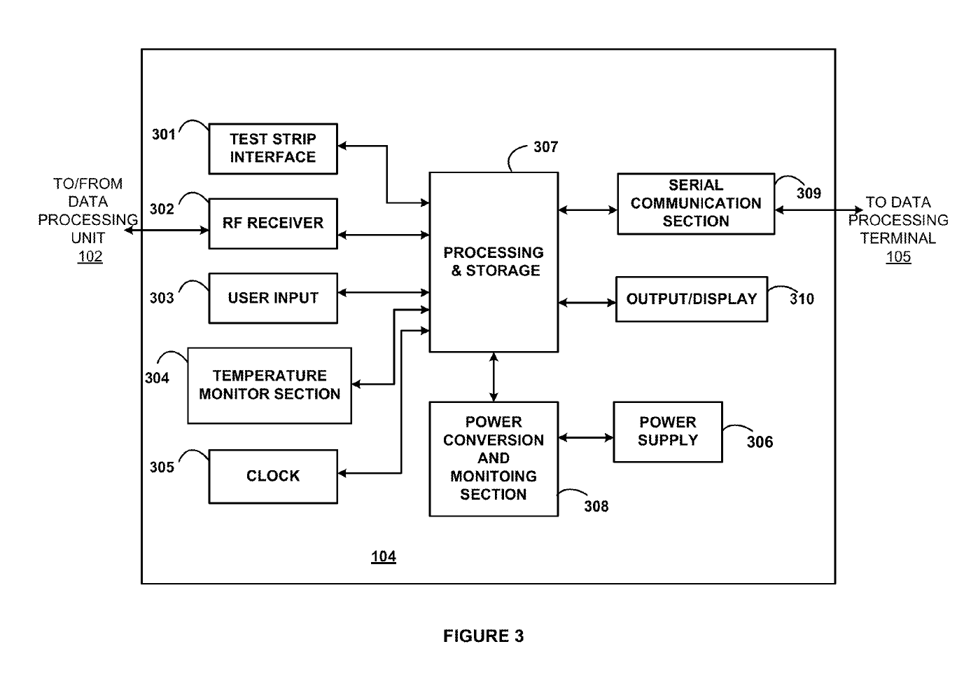

FIG. 3 is a block diagram of an embodiment of a receiver/monitor unit such as the primary receiver unit 104 of the data monitoring and management system shown in FIG. 1. The primary receiver unit 104 may include one or more of: a blood glucose test strip interface 301 for in vitro testing, an RF receiver 302, an input 303, a temperature detection section 304, and a clock 305, each of which is operatively coupled to a processing and storage section 307. The primary receiver unit 104 also includes a power supply 306 operatively coupled to a power conversion and monitoring section 308. Further, the power conversion and monitoring section 308 is also coupled to the receiver processor 307. Moreover, also shown are a receiver serial communication section 309, and an output 310, each operatively coupled to the processing and storage unit 307. The receiver may include user input and/or interface components or may be free of user input and/or interface components.

In certain embodiments having a test strip interface 301, the interface includes a glucose level testing portion to receive a blood (or other body fluid sample) glucose test or information related thereto. For example, the interface may include a test strip port to receive a glucose test strip. The device may determine the glucose level of the test strip, and optionally display (or otherwise notice) the glucose level on the output 310 of the primary receiver unit 104. Any suitable test strip may be employed, e.g., test strips that only require a very small amount (e.g., one microliter or less, e.g., 0.5 microliter or less, e.g., 0.1 microliter or less), of applied sample to the strip in order to obtain accurate glucose information, e.g. Freestyle® and Precision® blood glucose test strips from Abbott Diabetes Care Inc of Alameda, Calif.

Glucose information obtained by the in vitro glucose testing device may be used for a variety of purposes, computations, etc. For example, the information may be used to calibrate sensor 101 (however, calibration of the subject sensors may not be necessary), confirm results of the sensor 101 to increase the confidence thereof (e.g., in instances in which information obtained by sensor 101 is employed in therapy related decisions), etc. Exemplary blood glucose monitoring systems are described, e.g., in U.S. Pat. Nos. 6,071,391; 6,120,676; 6,338,790; and 6,616,819; and in U.S. application Ser. Nos. 11/282,001; and 11/225,659, the disclosures of which are herein incorporated by reference.

In further embodiments, the data processing unit 102 and/or the primary receiver unit 104 and/or the secondary receiver unit 106, and/or the data processing terminal/infusion section 105 may be configured to receive the blood glucose value from a wired connection or wirelessly over a communication link from, for example, a blood glucose meter. In further embodiments, a user manipulating or using the analyte monitoring system 100 (FIG. 1) may manually input the blood glucose value using, for example, a user interface (for example, a keyboard, keypad, voice commands, and the like) incorporated in the one or more of the data processing unit 102, the primary receiver unit 104, secondary receiver unit 106, or the data processing terminal/infusion section 105.

Additional detailed descriptions are provided in U.S. Pat. Nos. 5,262,035; 5,262,305; 5,264,104; 5,320,715; 5,593,852; 6,103,033; 6,134,461; 6,175,752; 6,560,471; 6,579,690; 6,605,200; 6,654,625; 6,746,582; and 6,932,894; and in U.S. Published Patent Application Nos. 2004/0186365, and and 2005/0182306 the disclosures of which are herein incorporated by reference.

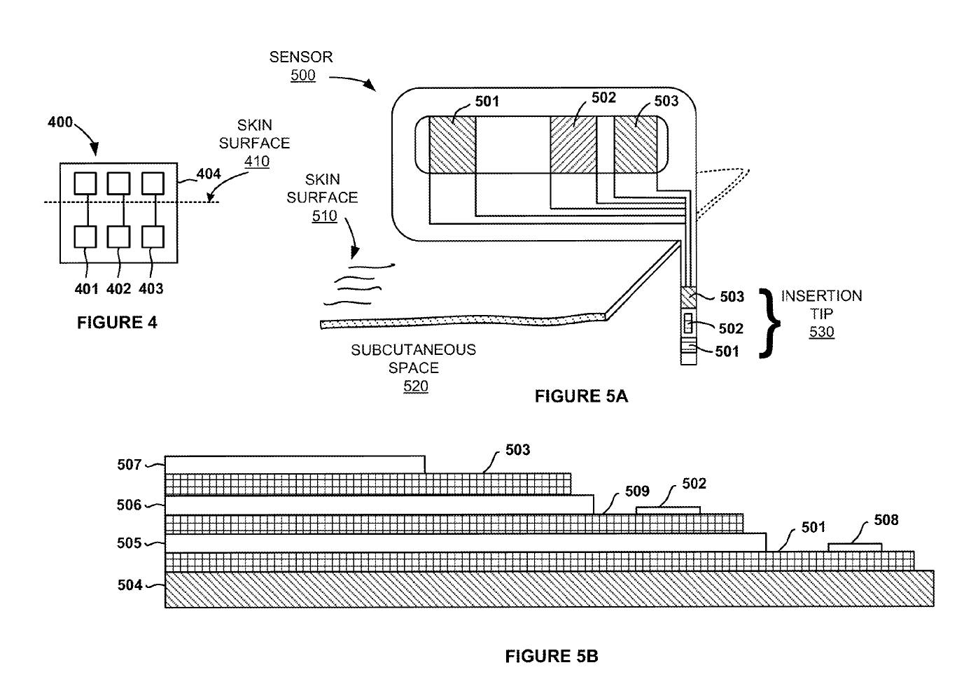

FIG. 4 schematically shows an embodiment of an analyte sensor 400 usable in the continuous analyte monitoring systems just described. This sensor embodiment includes electrodes 401, 402 and 403 on a base 404. Electrodes (and/or other features) may be applied or otherwise processed using any suitable technology, e.g., chemical vapor deposition (CVD), physical vapor deposition, sputtering, reactive sputtering, printing, coating, ablating (e.g., laser ablation), painting, dip coating, etching and the like. Suitable conductive materials include but are not limited to aluminum, carbon (such as graphite), cobalt, copper, gallium, gold, indium, iridium, iron, lead, magnesium, mercury (as an amalgam), nickel, niobium, osmium, palladium, platinum, rhenium, rhodium, selenium, silicon (e.g., doped polycrystalline silicon), silver, tantalum, tin, titanium, tungsten, uranium, vanadium, zinc, zirconium, mixtures thereof, and alloys, oxides, or metallic compounds of these elements.

The sensor may be wholly implantable in a user or may be configured so that only a portion is positioned within (internal) a user and another portion outside (external) a user. For example, the sensor 400 may include a portion positionable above a surface of the skin 410, and a portion positioned below the skin. In such embodiments, the external portion may include contacts (connected to respective electrodes of the second portion by traces) to connect to another device also external to the user such as a transmitter unit. While the embodiment of FIG. 4 shows three electrodes side-by-side on the same surface of base 404, other configurations are contemplated, e.g., fewer or greater electrodes, some or all electrodes on different surfaces of the base or present on another base, some or all electrodes stacked together, some or all electrodes twisted together (e.g., an electrode twisted around or about another or electrodes twisted together), electrodes of differing materials and dimensions, etc.

FIG. 5A shows a perspective view of an embodiment of an electrochemical analyte sensor 500 of the present invention having a first portion (which in this embodiment may be characterized as a major or body portion) positionable above a surface of the skin 510, and a second portion (which in this embodiment may be characterized as a minor or tail portion) that includes an insertion tip 530 positionable below the skin, e.g., penetrating through the skin and into, e.g., the subcutaneous space 520, in contact with the user's biofluid such as interstitial fluid. Contact portions of a working electrode 501, a reference electrode 502, and a counter electrode 503 are positioned on the portion of the sensor 500 situated above the skin surface 510. Working electrode 501, a reference electrode 502, and a counter electrode 503 are shown at the second section and particularly at the insertion tip 530. Traces may be provided from the electrode at the tip to the contact, as shown in FIG. 5A. It is to be understood that greater or fewer electrodes may be provided on a sensor. For example, a sensor may include more than one working electrode and/or the counter and reference electrodes may be a single counter/reference electrode, etc.

FIG. 5B shows a cross sectional view of a portion of the sensor 500 of FIG. 5A. The electrodes 501, 502 and 503 of the sensor 500 as well as the substrate and the dielectric layers are provided in a layered configuration or construction. For example, as shown in FIG. 5B, in one aspect, the sensor 500 (such as the sensor unit 101 FIG. 1), includes a substrate layer 504, and a first conducting layer 501 such as carbon, gold, etc., disposed on at least a portion of the substrate layer 504, and which may provide the working electrode. Also shown disposed on at least a portion of the first conducting layer 501 is a sensing component or layer 508, discussed in greater detail below. The area of the conducting layer covered by the sensing layer is herein referred to as the active area.

Referring to FIG. 5B, a first insulation layer such as a first dielectric layer 505 is disposed or layered on at least a portion of the first conducting layer 501, and further, a second conducting layer 502 may be disposed or stacked on top of at least a portion of the first insulation layer (or dielectric layer) 505, and which may provide the reference electrode. In one aspect, conducting layer 502 may include a layer of silver/silver chloride (Ag/AgCl), gold, etc. A second insulation layer 506 such as a dielectric layer in one embodiment may be disposed or layered on at least a portion of the conducting layer 509. Further, a third conducting layer 503 may provide the counter electrode 503. It may be disposed on at least a portion of the second insulation layer 506. Finally, a third insulation layer 507 may be disposed or layered on at least a portion of the third conducting layer 503. In this manner, the sensor 500 may be layered such that at least a portion of each of the conducting layers is separated by a respective insulation layer (for example, a dielectric layer). The embodiment of FIGS. 5A and 5B show the layers having different lengths. Some or all of the layers may have the same or different lengths and/or widths.

In addition to the electrodes, sensing layer and dielectric layers, sensor 500 may also include a temperature probe, a mass transport limiting layer, a biocompatible layer, and/or other optional components (none of which are illustrated). Each of these components enhances the functioning of and/or results from the sensor.

Substrate 504 may be formed using a variety of non-conducting materials, including, for example, polymeric or plastic materials and ceramic materials. It is to be understood that substrate includes any dielectric material of a sensor, e.g., around and/or in between electrodes of a sensor such as a sensor in the form of a wire wherein the electrodes of the sensor are wires that are spaced-apart by a substrate. In some embodiments, the substrate is flexible. For example, if the sensor is configured for implantation into a patient, then the sensor may be made flexible (although rigid sensors may also be used for implantable sensors) to reduce pain to the patient and damage to the tissue caused by the implantation of and/or the wearing of the sensor. A flexible substrate often increases the patient's comfort and allows a wider range of activities. Suitable materials for a flexible substrate include, for example, non-conducting plastic or polymeric materials and other non-conducting, flexible, deformable materials. Examples of useful plastic or polymeric materials include thermoplastics such as polycarbonates, polyesters (e.g., Mylar™ and polyethylene terephthalate (PET)), polyvinyl chloride (PVC), polyurethanes, polyethers, polyamides, polyimides, or copolymers of these thermoplastics, such as PETG (glycol-modified polyethylene terephthalate).

In other embodiments, the sensors, or at least a portion of the sensors, are made using a relatively rigid substrate, for example, to provide structural support against bending or breaking. Examples of rigid materials that may be used as the substrate include poorly conducting ceramics, such as aluminum oxide and silicon dioxide. One advantage of an implantable sensor having a rigid substrate is that the sensor may have a sharp point and/or a sharp edge to aid in implantation of a sensor without an additional insertion device. It will be appreciated that for many sensors and sensor applications, both rigid and flexible sensors will operate adequately. The flexibility of the sensor may also be controlled and varied along a continuum by changing, for example, the composition and/or thickness and/or width of the substrate (and/or the composition and/or thickness and/or width of one or more electrodes or other material of a sensor).

In addition to considerations regarding flexibility, it is often desirable that implantable sensors should have a substrate which is non-toxic. For example, the substrate may be approved by one or more appropriate governmental agencies or private groups for in vivo use.

Although the sensor substrate, in at least some embodiments, has uniform dimensions along the entire length of the sensor, in other embodiments, the substrate has a distal end or tail portion and a proximal end or body portion with different widths, respectively, as illustrated in FIG. 5A. In these embodiments, the distal end 530 of the sensor may have a relatively narrow width. For in vivo sensors which are implantable into the subcutaneous tissue or another portion of a patient's body, the narrow width of the distal end of the substrate may facilitate the implantation of the sensor. Often, the narrower the width of the sensor, the less pain the patient will feel during implantation of the sensor and afterwards.

For subcutaneously implantable sensors which are designed for continuous or semi-continuous monitoring of the analyte during normal activities of the patient, a tail portion or distal end of the sensor which is to be implanted into the patient may have a width of about 2 mm or less, e.g., about 1 mm or less, e.g., about 0.5 mm or less, e.g., about 0.25 mm or less, e.g., about 0.15 or less. However, wider or narrower sensors may be used. The proximal end of the sensor may have a width larger than the distal end to facilitate the connection between the electrode contacts and contacts on a control unit, or the width may be substantially the same as the distal portion.

The thickness of the substrate may be determined by the mechanical properties of the substrate material (e.g., the strength, modulus, and/or flexibility of the material), the desired use of the sensor including stresses on the substrate arising from that use, as well as the depth of any channels or indentations that may be formed in the substrate, as discussed below. The substrate of a subcutaneously implantable sensor for continuous or semi-continuous monitoring of the level of an analyte while the patient engages in normal activities may have a thickness that ranges from about 50 to about 500 μm, e.g., from about 100 to about 300 μm. However, thicker and thinner substrates may be used.

The length of the sensor may have a wide range of values depending on a variety of factors. Factors which influence the length of an implantable sensor may include the depth of implantation into the patient and the ability of the patient to manipulate a small flexible sensor and make connections between the sensor and the sensor control unit/transmitter. A subcutaneously implantable sensor of FIG. 5A may have an overall length ranging from about 0.3 to about 5 cm, however, longer or shorter sensors may be used. The length of the tail portion of the sensor (e.g., the portion which is subcutaneously inserted into the patient) is typically from about 0.25 to about 2 cm in length. However, longer and shorter portions may be used. All or only a part of this narrow portion may be subcutaneously implanted into the patient. The lengths of other implantable sensors will vary depending, at least in part, on the portion of the patient into which the sensor is to be implanted or inserted.

Electrodes 501, 502 and 503 are formed using conductive traces disposed on the substrate 504. These conductive traces may be formed over a smooth surface of the substrate or within channels formed by, for example, embossing, indenting or otherwise creating a depression in the substrate. The conductive traces may extend most of the distance along a length of the sensor, as illustrated in FIG. 5A, although this is not necessary. For implantable sensors, particularly subcutaneously implantable sensors, the conductive traces typically may extend close to the tip of the sensor to minimize the amount of the sensor that must be implanted.

The conductive traces may be formed on the substrate by a variety of techniques, including, for example, photolithography, screen printing, or other impact or non-impact printing techniques. The conductive traces may also be formed by carbonizing conductive traces in an organic (e.g., polymeric or plastic) substrate using a laser. A description of some exemplary methods for forming the sensor is provided in U.S. patents and applications noted herein, including U.S. Pat. Nos. 5,262,035; 6,103,033; 6,175,752; and 6,284,478, the disclosures of which are herein incorporated by reference.

Another method for disposing the conductive traces on the substrate includes the formation of recessed channels in one or more surfaces of the substrate and the subsequent filling of these recessed channels with a conductive material. The recessed channels may be formed by indenting, embossing, or otherwise creating a depression in the surface of the substrate. Exemplary methods for forming channels and electrodes in a surface of a substrate can be found in U.S. Pat. No. 6,103,033. The depth of the channels is typically related to the thickness of the substrate. In one embodiment, the channels have depths in the range of about 12.5 to about 75 μm, e.g., about 25 to about 50 μm.

The conductive traces are typically formed using a conductive material such as carbon (e.g., graphite), a conductive polymer, a metal or alloy (e.g., gold or gold alloy), or a metallic compound (e.g., ruthenium dioxide or titanium dioxide). The formation of films of carbon, conductive polymer, metal, alloy, or metallic compound are well-known and include, for example, chemical vapor deposition (CVD), physical vapor deposition, sputtering, reactive sputtering, printing, coating, and painting. In embodiments in which the conductive material is filled into channels formed in the substrate, the conductive material is often formed using a precursor material, such as a conductive ink or paste. In these embodiments, the conductive material is deposited on the substrate using methods such as coating, painting, or applying the material using a spreading instrument, such as a coating blade. Excess conductive material between the channels is then removed by, for example, running a blade along the substrate surface.

In certain embodiments, some or all of the electrodes 501, 502, 503 may be provided on the same side of the substrate 504 in the layered construction as described above, or alternatively, may be provided in a co-planar manner such that two or more electrodes may be positioned on the same plane (e.g., side-by side (e.g., parallel) or angled relative to each other) on the substrate 504. For example, co-planar electrodes may include a suitable spacing there between and/or include dielectric material or insulation material disposed between the conducting layers/electrodes. Furthermore, in certain embodiments, one or more of the electrodes 501, 502, 503 may be disposed on opposing sides of the substrate 504. In such double-sided sensor embodiments, the corresponding electrode contacts may be on the same or different sides of the substrate. For example, an electrode may be on a first side and its respective contact may be on a second side, e.g., a trace connecting the electrode and the contact may traverse through the substrate. Examples of double-sided sensors are disclosed in U.S. patent application Ser. Nos. 12/714,439 and 12/842,013, the disclosures of each of which are incorporated herein by reference.

As noted above, analyte sensors include an analyte-responsive enzyme to provide a sensing component or sensing layer 508 proximate to or on a surface of a working electrode in order to electrooxidize or electroreduce the target analyte on the working electrode. Some analytes, such as oxygen, can be directly electrooxidized or electroreduced, while other analytes, such as glucose and lactate, require the presence of at least one component designed to facilitate the electrochemical oxidation or reduction of the analyte. The sensing layer may include, for example, a catalyst to catalyze a reaction of the analyte and produce a response at the working electrode, an electron transfer agent to transfer electrons between the analyte and the working electrode (or other component), or both.

In certain embodiments, the sensing layer includes one or more electron transfer agents. Electron transfer agents that may be employed are electroreducible and electrooxidizable ions or molecules having redox potentials that are a few hundred millivolts above or below the redox potential of the standard calomel electrode (SCE). The electron transfer agent may be organic, organometallic, or inorganic. Examples of organic redox species are quinones and species that in their oxidized state have quinoid structures, such as Nile blue and indophenol. Examples of organometallic redox species are metallocenes such as ferrocene. Examples of inorganic redox species are hexacyanoferrate(III), ruthenium hexamine, etc.

In certain embodiments, electron transfer agents have structures or charges which prevent or substantially reduce the diffusional loss of the electron transfer agent during the period of time that the sample is being analyzed. For example, electron transfer agents include but are not limited to a redox species, e.g., bound to a polymer which can in turn be disposed on or near the working electrode. The bond between the redox species and the polymer may be covalent, coordinative, or ionic. Although any organic, organometallic or inorganic redox species may be bound to a polymer and used as an electron transfer agent, in certain embodiments the redox species is a transition metal compound or complex, e.g., osmium, ruthenium, iron, and cobalt compounds or complexes. It will be recognized that many redox species described for use with a polymeric component may also be used, without a polymeric component.

One type of polymeric electron transfer agent contains a redox species covalently bound in a polymeric composition. An example of this type of mediator is poly(vinylferrocene). Another type of electron transfer agent contains an ionically-bound redox species. This type of mediator may include a charged polymer coupled to an oppositely charged redox species. Examples of this type of mediator include a negatively charged polymer coupled to a positively charged redox species such as an osmium or ruthenium polypyridyl cation. Another example of an ionically-bound mediator is a positively charged polymer such as quaternized poly(4-vinyl pyridine) or poly(1-vinyl imidazole) coupled to a negatively charged redox species such as ferricyanide or ferrocyanide. In other embodiments, electron transfer agents include a redox species coordinatively bound to a polymer. For example, the mediator may be formed by coordination of an osmium or cobalt 2,2′-bipyridyl complex to poly(1-vinyl imidazole) or poly(4-vinyl pyridine).

Suitable electron transfer agents are osmium transition metal complexes with one or more ligands, each ligand having a nitrogen-containing heterocycle such as 2,2′-bipyridine, 1,10-phenanthroline, 1-methyl, 2-pyridyl biimidazole, or derivatives thereof. The electron transfer agents may also have one or more ligands covalently bound in a polymer, each ligand having at least one nitrogen-containing heterocycle, such as pyridine, imidazole, or derivatives thereof. Examples of an electron transfer agent include (a) a polymer or copolymer having pyridine or imidazole functional groups and (b) osmium cations complexed with two ligands, each ligand containing 2,2′-bipyridine, 1,10-phenanthroline, or derivatives thereof, the two ligands not necessarily being the same. Some derivatives of 2,2′-bipyridine for complexation with the osmium cation include but are not limited to 4,4′-dimethyl-2,2′-bipyridine and mono-, di-, and polyalkoxy-2,2′-bipyridines, such as 4,4′-dimethoxy-2,2′-bipyridine. Derivatives of 1,10-phenanthroline for complexation with the osmium cation include but are not limited to 4,7-dimethyl-1,10-phenanthroline and mono, di-, and polyalkoxy-1,10-phenanthrolines, such as 4,7-dimethoxy-1,10-phenanthroline. Polymers for complexation with the osmium cation include but are not limited to polymers and copolymers of poly(1-vinyl imidazole) (referred to as “PVI”) and poly(4-vinyl pyridine) (referred to as “PVP”). Suitable copolymer substituents of poly(1-vinyl imidazole) include acrylonitrile, acrylamide, and substituted or quaternized N-vinyl imidazole, e.g., electron transfer agents with osmium complexed to a polymer or copolymer of poly(1-vinyl imidazole).

Embodiments may employ electron transfer agents having a redox potential ranging from about −200 mV to about +200 mV versus the standard calomel electrode (SCE).

As mentioned above, the sensing layer may also include a catalyst which is capable of catalyzing a reaction of the analyte. The catalyst may also, in some embodiments, act as an electron transfer agent. When the analyte of interest is glucose, a catalyst such as a glucose oxidase, glucose dehydrogenase (e.g., pyrroloquinoline quinone (PQQ), dependent glucose dehydrogenase or oligosaccharide dehydrogenase, flavine adenine dinucleotide (FAD) dependent glucose dehydrogenase, or nicotinamide adenine dinucleotide (NAD) dependent glucose dehydrogenase) may be used. A lactate oxidase or lactate dehydrogenase may be used when the analyte of interest is lactate. Laccase may be used when the analyte of interest is oxygen or when oxygen is generated or consumed in response to a reaction of the analyte.

In certain embodiments, a catalyst may be attached to a polymer, cross linking the catalyst with another electron transfer agent which, as described above, may be polymeric. A second catalyst may also be used in certain embodiments. This second catalyst may be used to catalyze a reaction of a product compound resulting from the catalyzed reaction of the analyte. The second catalyst may operate with an electron transfer agent to electrolyze the product compound to generate a signal at the working electrode. Alternatively, a second catalyst may be provided in an interferent-eliminating layer to catalyze reactions that remove interferents.

Certain embodiments include a Wired Enzyme™ sensing layer (such as used in the FreeStyle Navigator® continuous glucose monitoring system by Abbott Diabetes Care Inc.) that works at a gentle oxidizing potential, e.g., a potential of about +40 mV. This sensing layer uses an osmium (Os)-based mediator designed for low potential operation and is stably anchored in a polymeric layer. Accordingly, in certain embodiments the sensing element is redox active component that includes (1) Osmium-based mediator molecules attached by stable (bidente) ligands anchored to a polymeric backbone, and (2) glucose oxidase enzyme molecules. These two constituents are crosslinked together.

In certain embodiments, the sensing system detects hydrogen peroxide to infer glucose levels. For example, a hydrogen peroxide-detecting sensor may be constructed in which a sensing layer includes enzyme such as glucose oxides, glucose dehydrogensae, or the like, and is positioned proximate to the working electrode. The sensing layer may be covered by one or more layers, e.g., a membrane that is selectively permeable to glucose. Once the glucose passes through the membrane, it may be oxidized by the enzyme and reduced glucose oxidase can then be oxidized by reacting with molecular oxygen to produce hydrogen peroxide.

Certain embodiments include a hydrogen peroxide-detecting sensor constructed from a sensing layer prepared by crosslinking two components together, for example: (1) a redox compound such as a redox polymer containing pendent Os polypyridyl complexes with oxidation potentials of about +200 mV vs. SCE, and (2) periodate oxidized horseradish peroxidase (HRP). Such a sensor functions in a reductive mode; the working electrode is controlled at a potential negative to that of the Os complex, resulting in mediated reduction of hydrogen peroxide through the HRP catalyst.

In another example, a potentiometric sensor can be constructed as follows. A glucose-sensing layer is constructed by crosslinking together (1) a redox polymer containing pendent Os polypyridyl complexes with oxidation potentials from about −200 mV to +200 mV vs. SCE, and (2) glucose oxidase. This sensor can then be used in a potentiometric mode, by exposing the sensor to a glucose containing solution, under conditions of zero current flow, and allowing the ratio of reduced/oxidized Os to reach an equilibrium value. The reduced/oxidized Os ratio varies in a reproducible way with the glucose concentration, and will cause the electrode's potential to vary in a similar way.

The components of the sensing layer may be in a fluid or gel that is proximate to or in contact with the working electrode. Alternatively, the components of the sensing layer may be disposed in a polymeric or sol-gel matrix that is proximate to or on the working electrode. Preferably, the components of the sensing layer are non-leachably disposed within the sensor. More preferably, the components of the sensor are immobilized within the sensor.

Examples of sensing layers that may be employed are described in U.S. patents and applications noted herein, including, e.g., in U.S. Pat. Nos. 5,262,035; 5,264,104; 5,543,326; 6,605,200; 6,605,201; 6,676,819; and 7,299,082, the disclosures of each of which are herein incorporated by reference.

Regardless of the particular components that make up a given sensing layer, a variety of different sensing layer configurations may be used. In certain embodiments, the sensing layer covers the entire working electrode surface, e.g., the entire width of the working electrode surface. In other embodiments, only a portion of the working electrode surface is covered by the sensing layer, e.g., only a portion of the width of the working electrode surface. Alternatively, the sensing layer may extend beyond the conductive material of the working electrode. In some cases, the sensing layer may also extend over other electrodes, e.g., over the counter electrode and/or reference electrode (or counter/reference is provided), and may cover all or only a portion thereof.

In other embodiments, the sensing layer is not deposited directly on the working electrode. Instead, the sensing layer may be spaced apart from the working electrode, and separated from the working electrode, e.g., by a separation layer. A separation layer may include one or more membranes or films or a physical distance. In addition to separating the working electrode from the sensing layer the separation layer may also act as a mass transport limiting layer and/or an interferent eliminating layer and/or a biocompatible layer.

In certain embodiments which include more than one working electrode, one or more of the working electrodes may not have a corresponding sensing layer, or may have a sensing layer which does not contain one or more components (e.g., an electron transfer agent and/or catalyst) needed to electrolyze the analyte. Thus, the signal at this working electrode may correspond to background signal which may be removed from the analyte signal obtained from one or more other working electrodes that are associated with fully-functional sensing layers by, for example, subtracting the signal.

A mass transport limiting layer or membrane, e.g., an analyte flux modulating layer, may be included with the sensor to act as a diffusion-limiting barrier to reduce the rate of mass transport of the analyte, for example, glucose or lactate, into the region around the working electrodes. The mass transport limiting layers are useful in limiting the flux of an analyte to a working electrode in an electrochemical sensor so that the sensor is linearly responsive over a large range of analyte concentrations. Mass transport limiting layers may include polymers and may be biocompatible. A mass transport limiting layer may provide many functions, e.g., biocompatibility and/or interferent-eliminating, etc.

A membrane may be formed by crosslinking in situ a polymer, modified with a zwitterionic moiety and a non-pyridine copolymer component. The modified polymer may be made from a precursor polymer containing heterocyclic nitrogen groups. For example, a precursor polymer may be polyvinylpyridine or polyvinylimidazole. Embodiments also include membranes that are made of a polyurethane, or polyether urethane, or chemically related material, or membranes that are made of silicone, and the like.

Optionally, another moiety or modifier that is either hydrophilic or hydrophobic, and/or has other desirable properties, may be used to “fine-tune” the permeability of the resulting membrane to an analyte of interest. Optional hydrophilic modifiers, such as poly(ethylene glycol), hydroxyl or polyhydroxyl modifiers, may be used to enhance the biocompatibility of the polymer or the resulting membrane.

The membrane may also be formed in situ by applying an alcohol-buffer solution of a crosslinker and a modified polymer over an enzyme-containing sensing layer and allowing the solution to cure for about one to two days or other appropriate time period. The crosslinker-polymer solution may be applied to the sensing layer by placing a droplet or droplets of the solution on the sensor, by dipping the sensor into the solution, or the like. Generally, the thickness of the membrane is controlled by the concentration of the solution, by the number of droplets of the solution applied, by the number of times the sensor is dipped in the solution, or by any combination of these factors. A membrane applied in this manner may have any combination of the following functions: (1) mass transport limitation, i.e., reduction of the flux of analyte that can reach the sensing layer, (2) biocompatibility enhancement, or (3) interferent reduction. Exemplary mass transport layers are described in U.S. patents and applications noted herein, including, e.g., in U.S. Pat. Nos. 5,593,852, 6,881,551, and 6,932,894, the disclosures of each of which are incorporated herein by reference.

In certain embodiments, a sensor may also include an active agent such as an anticlotting and/or antiglycolytic agent(s) disposed on at least a portion of a sensor that is positioned in a user. An anticlotting agent may reduce or eliminate the clotting of blood or other body fluid around the sensor, particularly after insertion of the sensor. Examples of useful anticlotting agents include heparin and tissue plasminogen activator (TPA), as well as other known anticlotting agents. Embodiments may include an antiglycolytic agent or precursor thereof. Examples of antiglycolytic agents are glyceraldehyde, fluoride ion, and mannose.

The electrochemical sensors of the present disclosure may employ any suitable measurement technique, e.g., may detect current, may employ potentiometry, etc. Techniques may include, but are not limited to amperometry, coulometry, and voltammetry. In some embodiments, sensing systems may be optical, colorimetric, and the like.

The analyte measurement systems with which the sensors are used may include an optional alarm system that, e.g., based on information from a processor, warns the patient of a potentially detrimental condition of the analyte. For example, if glucose is the analyte, an alarm system may warn a user of conditions such as hypoglycemia and/or hyperglycemia and/or impending hypoglycemia, and/or impending hyperglycemia. An alarm system may be triggered when analyte levels approach, reach or exceed a threshold value. An alarm system may also, or alternatively, be activated when the rate of change, or acceleration of the rate of change, in analyte level increase or decrease approaches, reaches or exceeds a threshold rate or acceleration. A system may also include system alarms that notify a user of system information such as battery condition, calibration, sensor dislodgment, sensor malfunction, etc. Alarms may be, for example, auditory and/or visual. Other sensory-stimulating alarm systems may be used including alarm systems which heat, cool, vibrate, or produce a mild electrical shock when activated.

The sensors may also be used in sensor-based drug delivery systems. The system may provide a drug to counteract the high or low level of the analyte in response to the signals from one or more sensors. Alternatively, the system may monitor the drug concentration to ensure that the drug remains within a desired therapeutic range. The drug delivery system may include one or more (e.g., two or more) sensors, a processing unit such as a transmitter, a receiver/display unit, and a drug administration system. In some cases, some or all components may be integrated in a single unit. A sensor-based drug delivery system may use data from the one or more sensors to provide necessary input for a control algorithm/mechanism to adjust the administration of drugs, e.g., automatically or semi-automatically. As an example, a glucose sensor may be used to control and adjust the administration of insulin from an external or implanted insulin pump.

Also, additional exemplary analyte monitoring systems are described in, for example, U.S. patent application Ser. Nos. 12/698,124, 12/698,129, and 12/807,278, the disclosures of each of which are incorporated herein by reference for all purposes. Moreover, detailed description of signal dropout detection or early signal attenuation detection and/or correction in analyte monitoring devices and systems is provided in U.S. patent application Ser. No. 12/363,712 and 61/243,989, the disclosures of each of which are incorporated herein by reference for all purposes.

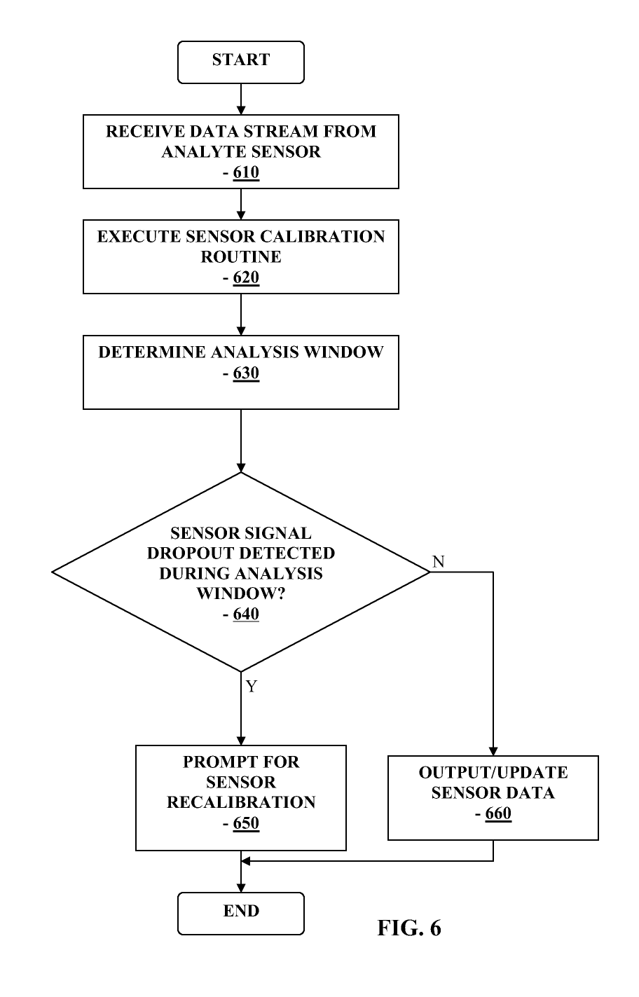

Referring back to the Figures, FIG. 6 is a flowchart illustrating signal dropout detection and processing routine in certain embodiments. Referring to FIG. 6, a data stream from the transcutaneously positioned analyte sensor in fluid contact with interstitial fluid, for example, is received (610). Optionally, the received data stream may be stored in whole or in part, in one or more memory or storage devices of the data processing unit 102 (FIG. 1), or the receiver unit 104/106, or the data processing terminal 105. Thereafter, when a scheduled or user initiated calibration event is triggered, the sensor calibration routine is executed (620). In one aspect, the calibration routine may be performed at a fixed or variable time schedule during the time period of the sensor use or wear (for example, 5 days, 7 days or more). In one aspect, calibration routine may include performing a blood glucose measurement using, for example, an in vitro blood glucose test strip.

Referring to FIG. 6, after calibrating the sensor for example, by the execution and completion of the sensor calibration routine, an analysis time window is determined or retrieved (from a memory or storage unit) (630). In one aspect, the analysis time window may be preprogrammed or programmable by the user or the healthcare provider, and include, for example, a 15 minute or 30 minute time duration measured from the completion of the calibration routine. Alternatively, other time durations less or greater than the 15 minute window or the 30 minute window, including for example, a 45 minute window, a 60 minute window, a 90 minute window, a 180 minute window or other suitable or programmable or programmed time windows, are contemplated as may be desired or suitable for the particular user or the patient. Moreover, the analysis time window may also be determined or measured to include the time period of the calibration routine execution such that the calibration routine time period is a subset of the analysis time period. After determining or retrieving the analysis time window, the received data stream from the sensor is analyzed or processed to determine the presence of a signal dropout condition (640). That is, in one embodiment, after the completion of the sensor calibration routine and the calibrated sensor data indicative of the monitored glucose level is reported or output to the user, the sensor data stream is analyzed to determine whether signal dropout has occurred.

In certain embodiments, signal dropout condition detection is performed continuously throughout the sensor wear time period. In certain embodiments, the signal dropout condition detection may be initiated prior to, concurrent with, and/or after the initiation of the calibration routine, during the calibration routine, or after the completion of the calibration routine. More specifically, in certain embodiments, the signal dropout condition detection is performed continuously and substantially simultaneously upon receipt of data from the analyte sensor. As such, a patient or user may be notified of a potential signal dropout condition in real-time or near real-time and, if needed, take corrective action, such as recalibration, replacement of the sensor, or change the sensor's insertion location.

In one embodiment, the signal dropout condition may be declared or detected when two or more consecutive data points from the sensor data stream exhibit a variance, deviation or difference (relative to each other) that is greater or exceeds a predetermined threshold level. For example, if a transition of signal magnitude between adjacent sensor data points in the received data stream indicating a magnitude change greater than 15% or greater relative to each other is detected, a signal dropout condition may be declared to indicate that the variation in the signal magnitude change is attributable to one or more parameters or conditions other than the monitored analyte level. In other words, the received sensor signal is determined to be an inaccurate indication of the monitored analyte level. While a magnitude change of greater than 15% is described above, in accordance with the embodiments of the present disclosure, larger or smaller variation percentages, such as 30% change in magnitude, 10% change in magnitude, and the like, may be programmed or set as the predetermined threshold level to declare or confirm the presence of a signal dropout condition.

Referring back to FIG. 6, when the signal dropout condition is detected, a prompt or output indication is generated to notify the user or the patient to perform sensor recalibration routine (650). That is, when the signal dropout condition is detected during the analysis time window, the executed sensor calibration routine (620) is considered to include an error or is otherwise unacceptable or inaccurate, and therefore, the user is prompted to recalibrate the sensor. On the other hand, if the signal dropout condition is not detected during the analysis time window, then one or more of the calibrated sensor data and/or an indication of the sensor data stream is output (including, for example, a graphical display, an audible or vibratory output, a numerical output or one or more combinations thereof) or updated for presentation to the user or the patient (660).

In one aspect, the outputted information may include a numerical glucose value associated with the real time monitored glucose level in units of mg/dL, for example. Additionally, in still a further aspect, the outputted information may include a graphical representation (such as a line graph) of the monitored glucose level spanning a predetermined time period (which may include the entire sensor life or a portion thereof), or a directional indicator of the direction in which the monitored analyte level is changing as well as the rate of change of the monitored analyte level.

Referring still to FIG. 6, when the signal dropout condition is detected during the determined analysis time window and the user is prompted to perform recalibration routine (650), the associated real-time data stream from the analyte sensor may continue to be output to the user with an indication that recalibration routine is in progress, or alternatively, with an indication that the signal dropout condition has been detected. Further, in certain embodiments, the output or presentation of information associated with the monitored analyte level based on the received data stream may be temporarily suspended until the recalibration routine has been successfully completed. The recalibration routine in one aspect may include obtaining a reference data point using for example, an in vitro blood sample obtained from a finger stick test using an in vitro test strip and a blood glucose meter. That is, the recalibration routine may include the same or similar steps as the calibration routine performed.

In certain embodiments, when signal dropout condition is detected in the analyte monitoring system 100 during, prior to, or following a calibration routine, the user or the patient is prompted or instructed to perform recalibration, and the previously initiated, executed or completed calibration routine is flagged or identified as a failed calibration routine. Accordingly, by analyzing the data stream from the sensor, a change in the sensor signal level determined before, during or after the calibration routine may be used to identify an unsuitable calibration condition retrospectively. In this manner, when sensor signal error is detected, certain of the routines associated with the sensor signal processing such as calibration that were performed within the analysis time window are deemed to contain error, and as such, the user or the patient may be required or prompted to perform the identified routines again to increase accuracy and minimize system errors.

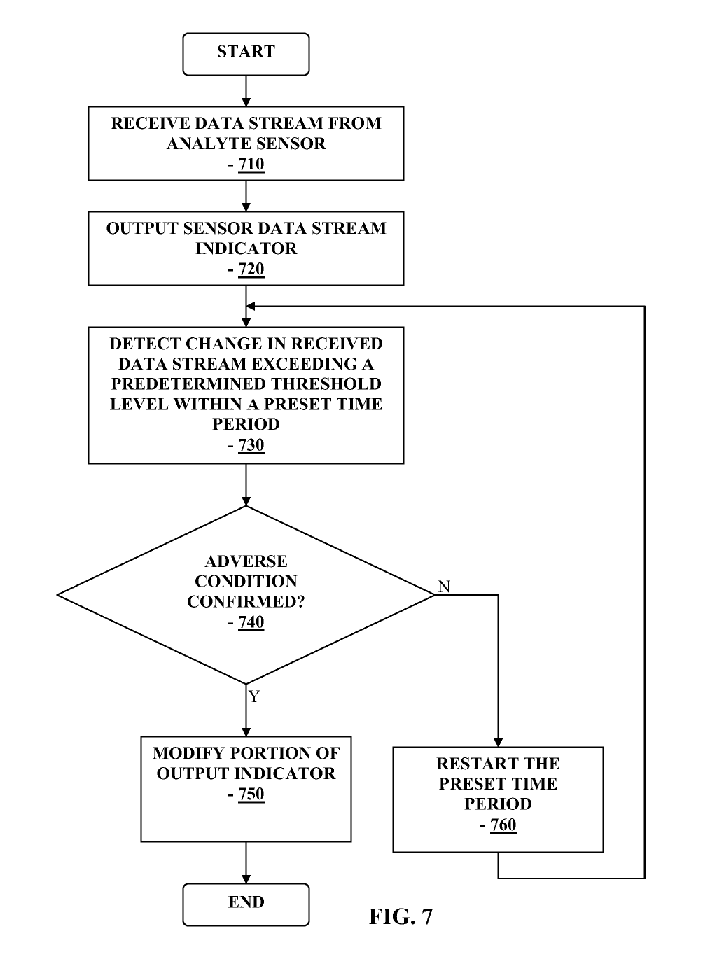

FIG. 7 is a flowchart illustrating signal dropout detection and processing routine in certain embodiments. Referring to FIG. 7, when the data stream from the analyte sensor is received (710), one or more indicators associated with the received data stream is output (720) including, for example, the corresponding monitored analyte level information. In one aspect, the output data stream may include processed or filtered signals performed on the received data stream to remove signal artifacts, noise, and other variables that may be a source of error or inaccuracy. Thereafter, when a change in the received data stream exceeding a predetermined threshold level within a preset time period is detected (730), it is determined whether the detected change in the received data stream is a confirmation of the presence of an adverse condition such as a signal dropout condition (740).

If the adverse condition is confirmed, as shown in FIG. 7, a portion of the output indicator is modified to reflect the detection of the confirmed adverse condition (750). On the other hand, if the detected change in the received data stream is not a confirmed adverse condition, then the preset time period is restarted (immediately or after the expiration of the preset time period during which the change in the data stream is detected) (760) and the routine returns to monitor for the detection of a change in the received data stream exceeding the predetermined threshold level within the newly started preset time window.

Referring back to FIG. 7, the output sensor data stream indicator may include a graphical representation of the received sensor data stream over a certain time period such as from the initial positioning of the sensor in fluid contact with the interstitial fluid of the user or the patient. Thus, in one aspect, when the adverse signal condition is confirmed during the sensor use or wear, the portion of the graphical representation during the preset time window in which the change in the received data stream is detected and the adverse condition confirmed is modified to indicate potential error in the output graphical representation. For example, in certain embodiments, a line graph with a preselected default color, thickness, or format may be used to present the monitored glucose level based on the data stream from the sensor.

When, during the preset time window such as, for example, a 15 minute window, the adverse signal condition is detected and confirmed, that portion of the line graph may be modified with a different output indication (such as a different color, different thickness or a different format). Accordingly, the user may readily and easily be able to determine the portion of the graphical representation of the associated monitored analyte level during which the adverse condition was confirmed (and thus, likely reject or not rely on the information). Additionally, while a 15 minute time window is described above as the preset time window, in accordance with the embodiments of the present disclosure, other time windows such as, but not limited to 5 minute window, 10 minute window, 30 minute window, 45 minute window or 60 minute window, for example, are contemplated. Additionally, the user or the healthcare provider may optionally adjust the preset time window such that it is programmable or adjustable from a default setting, for example.

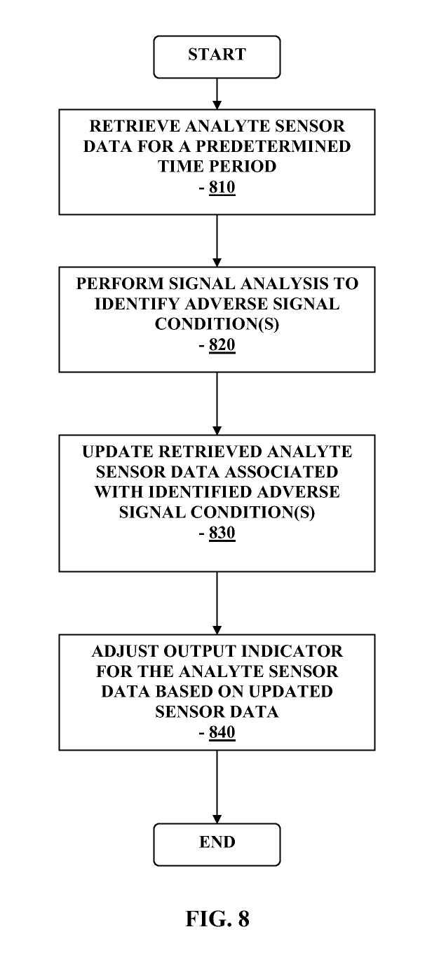

FIG. 8 is a flowchart illustrating signal dropout detection and processing routine in certain embodiments. Referring now to FIG. 8, in certain embodiments, retrospective analysis of the data stream from the sensor is implemented. For example, as shown in FIG. 8, after the analyte sensor data is retrieved for a predetermined time period (which for example, may include the entire duration of one or more sensor use or a subset thereof) (810), signal analysis is performed on the retrieved sensor data to identify one or more adverse signal conditions (820) based on, for example, the conditions of a signal dropout as described above. After performing the signal analysis to identify the adverse signal conditions, the retrieved analyte sensor data associated with the identified adverse signal conditions are updated (830).

In one aspect, updating the retrieved sensor data may include adjusting or modifying the corresponding sensor data values to compensate for the adverse conditions. The adjustment or modification to the retrieved sensor data may include, for example, performing a linear or non-linear line fit, a regression analysis, an autoregression analysis, an averaging, or an executing of a complex signal correction algorithm or routine based on one or more of a rate of change information of the sensor data, contemporaneous sensor data values that are not associated with the identified adverse signal conditions, directional change of the sensor data values, and the like.

Referring back to FIG. 8, in certain embodiments, after updating the retrieved analyte sensor data associated with the identified adverse signal condition(s), an output indication or a portion thereof (such as, for example, a graphical representation of the sensor data indicating a monitored analyte level) is adjusted, updated, highlighted, flagged, modified, replaced, or masked based on the updated sensor data (840). In this manner, data streams received from one or more analyte sensors over a given time period that have been collected or stored may be analyzed to identify signal dropout conditions that are not associated with the variation in the monitored analyte level, and also, to adjust or correct the stored sensor data to improve accuracy and to allow proper therapeutic actions to be taken based on the information with minimized error.

Such routines as described above in conjunction with FIG. 8 may be performed using a data analysis tool or software resident on a computer terminal or the data processing terminal 105 (FIG. 1) of the analyte monitoring system 100. Within the scope of the present disclosure, the receiver unit 104/106 may also include capabilities to perform the routines described above in conjunction with FIG. 8 in addition to FIGS. 6 and 7.

In certain embodiments, the retrospective dropout detection analysis described above in conjunction with FIG. 8, may be performed using a data management system including, for example, a personal computer (PC) or other computing or logic processing or programmed/programmable routine(s) execution system, such as the data processing terminal 105 (FIG. 1), for example. After completion of the retrospective analysis, the data, as modified by the correction, or updated based on the detected signal dropouts, may be displayed on a display device, such as a monitor or other screen, of the data management system. In certain embodiments, the modified, corrected or updated sensor data may be displayed in a graphical or chart representation overlaid onto the original received data for the time period in which the retrospective analysis was performed. As such, a patient or other user may be able to conveniently see the corrections to the data made by the retrospective analysis and signal dropout detection.

In certain embodiments, the corrections or modifications to the received sensor data associated with the signal dropout detection analysis may be individually identified by, for example, markers, flags, or highlighted areas of the display. In certain embodiments, both the real-time data and the modified, corrected, or updated sensor data may be stored in a memory of the data management system, or in alternative embodiments, only the modified, corrected, or updated data is stored. In certain embodiment, the data management system may include receiver unit 104/106 or data processing terminal 105 of analyte monitoring system 100 (FIG. 1).

In certain embodiments, the retrospective analysis may be performed using one or more time periods that are suitable for data analysis which may coincide with the sensor life, extend beyond the sensor life, or include a portion of the sensor life. For example, in certain embodiments, a 7 day time period for retrospective analysis may be performed in order to give a patient a week long analysis of the frequency of detected dropouts or a determination of a pattern of particular times of day where detected dropouts are more likely to occur. In certain embodiments, a shorter time period for the retrospective analysis may be used, such as a 12 hour time period. A shorter time period retrospective analysis may be useful, for example, to determine dropout frequency during a particular time of day, such as nighttime, pre-meal, etc. Based on the determined frequency or pattern of detected signal dropouts, the data management system may provide recommendations for modification of use, such as, for example, recommendations to replace the sensor, to change the sensor insertion location, the time of day during which to replace the sensor, or the time period(s) suitable for performing calibration of the sensor, if necessary. Other time periods for the retrospective signal dropout analysis may also be utilized including, but not limited to, a 24 hour analysis, a 48 hour analysis, a 72 hour analysis, a 7 day analysis, a 14 day analysis, or a 30 day analysis, for example.

In this manner, in accordance with the various embodiments of the present disclosure, real time or retrospective analysis of sensor data may be performed to detect or identify the presence of a signal dropout condition, and to alert or notify the user or the healthcare provider, initiate or execute previously executed functions or routines or otherwise correct or compensate for the errors associated with the signal dropout condition to improve accuracy of the reported or monitored glucose levels presented to the user or the healthcare provider and/or used for further analysis including data processing for therapy management.

In one embodiment, a method may comprise receiving a plurality of signals from a transcutaneously positioned analyte sensor, receiving a reference data, calibrating the analyte sensor based on the received reference data to generate calibrated sensor data, detecting a change in the level of the received plurality of signals from the analyte sensor exceeding a predetermined threshold level within a preset time period after calibrating the analyte sensor, and generating an output signal based on the detected change.

The received reference data may be a blood glucose measurement.

Moreover, the method may include performing an in vitro test to obtain the blood glucose measurement.

Calibrating the analyte sensor may include determining a calibration factor based on the received reference data and a substantially time corresponding signal received from the analyte sensor.

Determining the calibration factor may include matching the received reference data to the substantially time corresponding sensor signal.

The calibration factor may include a sensitivity ratio.

Calibrating the analyte sensor may include applying the calibration factor to the received plurality of signals from the sensor.

The detected change in the level of the received plurality of signals from the sensor may be associated with a signal dropout condition.

The predetermined threshold level may include a variation in the level of at least two adjacent signals from the received plurality of signals exceeding approximately 5%.

The preset time period may include approximately one hour or less, 45 minutes or less, 30 minutes or less, 15 minutes or less, 10 minutes or less, or 5 minutes or less.

The generated output may include a prompt to recalibrate the sensor.

In one aspect, the method may include masking the calibrated sensor data.

Masking the calibrated sensor data may include disabling the output of the calibrated sensor data.

The reference data may be received from the transcutaneously positioned analyte sensor.

In another embodiment, a method may comprise receiving a plurality of signals from a transcutaneously positioned analyte sensor; outputting an indicator associated with the received plurality of signals from the sensor; detecting a change in the level of the received plurality of signals from the analyte sensor exceeding a predetermined threshold level within a preset time period; confirming an adverse condition based on the detected change in the level of the received plurality of signals from the sensor; and modifying a portion of the outputted indicator based on the confirmed adverse signal condition.

The adverse condition may include a signal dropout condition.

The indicator may include a graphical representation associated with the received plurality of signals from the sensor.

Modifying the portion of the outputted indicator may include identifying a subset of the received plurality of signals from the sensor associated with the confirmed adverse condition, and modifying the portion of the outputted indicator corresponding to the subset of the received plurality of signals from the sensor.

In another embodiment, an apparatus may comprise one or more processors; and a memory operatively coupled to the one or more processors for storing instructions which, when executed by the one or more processors, causes the one or more processors to receive a plurality of signals from a transcutaneously positioned analyte sensor, receive a reference data, calibrate the analyte sensor based on the received reference data to generate calibrated sensor data, detect a change in the level of the received plurality of signals from the analyte sensor exceeding a predetermined threshold level within a preset time period after calibrating the analyte sensor, and generate an output signal based on the detected change.

The received reference data may be a blood glucose measurement from an in vitro test strip.

The memory operatively coupled to the one or more processors for storing instructions which, when executed by the one or more processors, may cause the one or more processors to determine a calibration factor based on the received reference data and a substantially time corresponding signal received from the analyte sensor.

The memory operatively coupled to the one or more processors for storing instructions which, when executed by the one or more processors, may cause the one or more processors to match the received reference data to the substantially time corresponding sensor signal.

The calibration factor may include a sensitivity ratio.

The memory operatively coupled to the one or more processors for storing instructions which, when executed by the one or more processors, may cause the one or more processors to apply the calibration factor to the received plurality of signals from the sensor.

The detected change in the level of the received plurality of signals from the sensor may be associated with a signal dropout condition.

The predetermined threshold level may include a variation in the level of at least two adjacent signals from the received plurality of signals exceeding approximately 5%.

The preset time period may include approximately 15 minutes or less.

The generated output may include a prompt to recalibrate the sensor.

The memory operatively coupled to the one or more processors for storing instructions which, when executed by the one or more processors, may cause the one or more processors to mask the calibrated sensor data.

The memory operatively coupled to the one or more processors for storing instructions which, when executed by the one or more processors, may cause the one or more processors to disable the output of the calibrated sensor data.

The reference data may be received from the transcutaneously positioned analyte sensor.

In yet another embodiment, an apparatus may comprise one or more processors; an output unit operatively coupled to the one or more processors; and a memory operatively coupled to the one or more processors for storing instructions which, when executed by the one or more processors, causes the one or more processors to receive a plurality of signals from a transcutaneously positioned analyte sensor, output an indicator associated with the received plurality of signals from the sensor to the output unit, detect a change in the level of the received plurality of signals from the analyte sensor exceeding a predetermined threshold level within a preset time period, confirm an adverse condition based on the detected change in the level of the received plurality of signals from the sensor, and modify a portion of the outputted indicator based on the confirmed adverse signal condition.

The adverse condition may include a signal dropout condition.

The indicator may include a graphical representation associated with the received plurality of signals from the sensor.

The memory operatively coupled to the one or more processors for storing instructions which, when executed by the one or more processors, may cause the one or more processors to identify a subset of the received plurality of signals from the sensor associated with the confirmed adverse condition, and to modify the portion of the outputted indicator corresponding to the subset of the received plurality of signals from the sensor.

As for other details of the present invention, materials and alternate related configurations may be employed as within the level of those with skill in the relevant art. The same may hold true with respect to method-based aspects of the invention in terms of additional acts as commonly or logically employed. In addition, though the invention has been described in reference to several examples, optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. Any number of the individual parts or subassemblies shown may be integrated in their design. Such changes or others may be undertaken or guided by the principles of design for assembly.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in the appended claims, the singular forms “a,” “an,” “said,” and “the” include plural referents unless the specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Without the use of such exclusive terminology, the term “comprising” in the claims shall allow for the inclusion of any additional element—irrespective of whether a given number of elements are enumerated in the claim, or the addition of a feature could be regarded as transforming the nature of an element set forth n the claims. Stated otherwise, unless specifically defined herein, all technical and scientific terms used herein are to be given as broad a commonly understood meaning as possible while maintaining claim validity.

Various other modifications and alterations in the structure and method of operation of the embodiments of the present disclosure will be apparent to those skilled in the art without departing from the scope and spirit of the present disclosure. Although the present disclosure has been described in connection with certain embodiments, it should be understood that the present disclosure as claimed should not be unduly limited to such embodiments. It is intended that the following claims define the scope of the present disclosure and that structures and methods within the scope of these claims and their equivalents be covered thereby.

What is claimed is:

1. A method, comprising:
receiving a plurality of signals from a transcutaneously positioned analyte sensor;
receiving a reference data;
calibrating the analyte sensor based on the received reference data to generate calibrated sensor data;
detecting a change in the level of the received plurality of signals from the analyte sensor exceeding a predetermined threshold level within a preset time period after calibrating the analyte sensor; and
generating an output signal based on the detected change.

2. The method of claim 1 wherein the received reference data is a blood glucose measurement.

3. The method of claim 2 including performing an in vitro test to obtain the blood glucose measurement.

4. The method of claim 1 wherein calibrating the analyte sensor includes determining a calibration factor based on the received reference data and a substantially time corresponding signal received from the analyte sensor.

5. The method of claim 4 wherein determining the calibration factor includes matching the received reference data to the substantially time corresponding sensor signal.

6. The method of claim 4 wherein the calibration factor includes a sensitivity ratio.

7. The method of claim 4 wherein calibrating the analyte sensor includes applying the calibration factor to the received plurality of signals from the sensor.

8. The method of claim 1 wherein the detected change in the level of the received plurality of signals from the sensor is associated with a signal dropout condition.

9. The method of claim 1 wherein the predetermined threshold level includes a variation in the level of at least two adjacent signals from the received plurality of signals exceeding approximately 5%.

10. The method of claim 1 wherein the preset time period includes approximately 60 minutes or less, 45 minutes or less, 30 minutes or less, 15 minutes or less, 10 minutes or less, or 5 minutes or less.

11. The method of claim 1 wherein the generated output includes a prompt to recalibrate the sensor.

12. The method of claim 1 including masking the calibrated sensor data.

13. The method of claim 12 wherein masking the calibrated sensor data includes disabling the output of the calibrated sensor data.

14. The method of claim 1 wherein the reference data is received from the transcutaneously positioned analyte sensor.

15. A method, comprising:
receiving a plurality of signals from a transcutaneously positioned analyte sensor;
outputting an indicator associated with the received plurality of signals from the sensor;
detecting a change in the level of the received plurality of signals from the analyte sensor exceeding a predetermined threshold level within a preset time period;
confirming an adverse condition based on the detected change in the level of the received plurality of signals from the sensor; and
modifying a portion of the outputted indicator based on the confirmed adverse signal condition.

16. The method of claim 15 wherein the adverse condition includes a signal dropout condition.

17. The method of claim 15 wherein the indicator includes a graphical representation associated with the received plurality of signals from the sensor.

18. The method of claim 15 wherein modifying the portion of the outputted indicator includes identifying a subset of the received plurality of signals from the sensor associated with the confirmed adverse condition, and modifying the portion of the outputted indicator corresponding to the subset of the received plurality of signals from the sensor.

19. An apparatus, comprising:
one or more processors; and
a memory operatively coupled to the one or more processors for storing instructions which, when executed by the one or more processors, causes the one or more processors to receive a plurality of signals from a transcutaneously positioned analyte sensor, receive a reference data, calibrate the analyte sensor based on the received reference data to generate calibrated sensor data, detect a change in the level of the received plurality of signals from the analyte sensor exceeding a predetermined threshold level within a preset time period after calibrating the analyte sensor, and generate an output signal based on the detected change.

20. The apparatus of claim 19 wherein the received reference data is a blood glucose measurement from an in vitro test strip.

21. The apparatus of claim 19 wherein the memory operatively coupled to the one or more processors for storing instructions which, when executed by the one or more processors, causes the one or more processors to determine a calibration factor based on the received reference data and a substantially time corresponding signal received from the analyte sensor.

22. The apparatus of claim 21 wherein the memory operatively coupled to the one or more processors for storing instructions which, when executed by the one or more processors, causes the one or more processors to match the received reference data to the substantially time corresponding sensor signal.

23. The apparatus of claim 21 wherein the calibration factor includes a sensitivity ratio.

24. The apparatus of claim 21 wherein the memory operatively coupled to the one or more processors for storing instructions which, when executed by the one or more processors, causes the one or more processors to apply the calibration factor to the received plurality of signals from the sensor.

25. The apparatus of claim 19 wherein the detected change in the level of the received plurality of signals from the sensor is associated with a signal dropout condition.

26. The apparatus of claim 19 wherein the predetermined threshold level includes a variation in the level of at least two adjacent signals from the received plurality of signals exceeding approximately 5%.

27. The apparatus of claim 19 wherein the preset time period includes approximately 60 minutes or less, 45 minutes or less, 30 minutes or less, 15 minutes or less, 10 minutes or less, or 5 minutes or less.

28. The apparatus of claim 19 wherein the generated output includes a prompt to recalibrate the sensor.

29. The apparatus of claim 19 wherein the memory operatively coupled to the one or more processors for storing instructions which, when executed by the one or more processors, causes the one or more processors to mask the calibrated sensor data.

30. The apparatus of claim 29 wherein the memory operatively coupled to the one or more processors for storing instructions which, when executed by the one or more processors, causes the one or more processors to disable the output of the calibrated sensor data.

31. The apparatus of claim 19 wherein the reference data is received from the transcutaneously positioned analyte sensor.

32. An apparatus, comprising:
one or more processors;
an output unit operatively coupled to the one or more processors; and
a memory operatively coupled to the one or more processors for storing instructions which, when executed by the one or more processors, causes the one or more processors to receive a plurality of signals from a transcutaneously positioned analyte sensor, output an indicator associated with the received plurality of signals from the sensor to the output unit, detect a change in the level of the received plurality of signals from the analyte sensor exceeding a predetermined threshold level within a preset time period, confirm an adverse condition based on the detected change in the level of the received plurality of signals from the sensor, and modify a portion of the outputted indicator based on the confirmed adverse signal condition.

33. The apparatus of claim 32 wherein the adverse condition includes a signal dropout condition.

34. The apparatus of claim 33 wherein the indicator includes a graphical representation associated with the received plurality of signals from the sensor.

35. The apparatus of claim 32 wherein the memory operatively coupled to the one or more processors for storing instructions which, when executed by the one or more processors, causes the one or more processors to identify a subset of the received plurality of signals from the sensor associated with the confirmed adverse condition, and to modify the portion of the outputted indicator corresponding to the subset of the received plurality of signals from the sensor.
